(19) United States
(12) Patent Application Publication
(10) Pub. No.: US 2002/0166143 A1
(43) Pub. Date: Nov. 7, 2002

(54) Sclerotinia-inducible genes and promoters and their uses

(75) Inventors: Zhongmeng Bao, Urbandale, IA (US); Guihua Lu, Urbandale, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 09/923,844

(22) Filed: Aug. 7, 2001

Related U.S. Application Data
(60) Provisional application No. 60/224,603, filed on Aug. 11, 2000.

(51) Int. Cl. A01H 5/00; C07H 21/04; C12N 15/82; C12N 5/04

(52) U.S. Cl. 800/279; 435/468; 435/320.1; 435/419; 536/23.6

(57) ABSTRACT

Compositions and methods to aid in protecting plants from invading pathogenic organisms are provided. The compositions of the invention comprise anti-pathogenic genes, including their promoters, and proteins encoded by the anti-pathogenic genes. The compositions find use in methods for reducing or eliminating damage to plants caused by plant pathogens. Transformed plants, plant cells, tissues, and seed are also provided having enhanced disease resistance.

### CROSS-REFERENCE TO RELATED APPLICATIONS

This application claims the benefit of U.S. Provisional Application Serial No. 60/224,603, filed Aug. 11, 2000, which is hereby incorporated in its entirety by reference herein.

### FIELD OF THE INVENTION

The invention relates to nucleotide sequences and proteins for anti-pathogenic agents and their uses, particularly the genetic manipulation of plants with genes and promoters that enhance disease resistance.

### BACKGROUND OF THE INVENTION

Among the causal agents of infectious diseases of crop plants, phytopathogenic fungi play the dominant role. Phytopathogenic fungi can cause devastating epidemics such as the potato blight which led to the Irish potato famine. Phytopathogenic fungi also contribute to the persistent and significant annual crop yield losses that have made fungal pathogens a serious economic factor. Further, feed material infected with fungi poses a greater threat of teratogenic effects to animals that consume it.

All flowering plant species are attacked by pathogenic fungi. Fungal microorganisms have evolved strategies and mechanisms to parasitize plants, including invasion of plant tissue, optimization of growth in the plant, and propagation in or on the plant. Invasion of plant tissue by bacteria and viruses as well as some opportunistic fungal parasites often depends on the presence of natural openings or wounds. In contrast, many true phytopathogenic fungi have evolved mechanisms such as hydrolytic enzymes to actively traverse the plants' outer structural barriers, such as the cuticle and the epidermal cell wall. Once established in a plant, fungal diseases can rapidly spread throughout an entire field of a crop, and can even spread across broad geographical regions to an entire crop.

Despite the large number of microorganisms capable of causing disease, most plants are resistant to any given pathogen. The defense mechanisms utilized by plants can take many different forms, ranging from non-pathogen-specific defenses such as passive mechanical or preformed chemical barriers to more active and specific responses that provide host- or variety-specific resistance.

A hypersensitive response (HR) that is elaborated in response to invasion by all classes of pathogens is the most common feature associated with active host resistance. In most cases, activation of the HR leads to the death of cells at the infection site, which results in the restriction of the pathogen to small areas immediately surrounding the initially infected cells. At the whole-plant level, the HR is manifested as small necrotic lesions. Because the number of cells affected by the HR is only a small fraction of the total in the plant, this localized cell death response contributes to the survival of plants undergoing pathogen attack.

In plants, robust defense responses to invading phytopathogens often conform to a gene-for-gene relationship. Resistance to a pathogen is only observed when the pathogen carries a specific avirulence (avr) gene and the plant carries a corresponding resistance (R) gene. Because avr-R gene-for-gene relationships are observed in many plant-pathogen systems and are accompanied by a characteristic set of defense responses, a common molecular mechanism underlying avr-R gene-mediated resistance has been postulated. Thus, disease resistance results from the expression of a resistance gene in the plant and a corresponding avirulence gene in the pathogen and is often associated with the rapid, localized cell death characteristic of the hypersensitive response. R genes that respond to specific bacteria, fungal, or viral pathogens have been isolated from a variety of plant species and several appear to encode cytoplasmic proteins.

Other genes expressed in the plant defense response include “pathogenesis-related” (“PR”) genes, which perform a variety of functions to assist in preventing further infection. The PR genes include glucanases and chitinases, which attack the cell walls of fungi. Other PR genes and other genes expressed in response to pathogen attack are thought to perform their defensive roles by more indirect means. For example, products of such genes may be involved in regulation of the disease resistance signal production pathway. Silva et al. (1999), Mol. Plant Microbe Interact. 12(12): 1053-63.

The development of new strategies to control diseases is the primary purpose of research on plant-pathogen interactions and the pathogen response. Research efforts include, for example, the identification of essential pathogen virulence factors and the development of means to block them, or the transfer of resistance genes into crop plants from unrelated species. An additional benefit is a better understanding of the physiology of the healthy plant through a study of the metabolic disturbances caused by plant pathogens.

### SUMMARY OF THE INVENTION

Anti-pathogenic compositions and methods for their use are provided. The compositions comprise anti-pathogenic proteins and their corresponding gene sequences and regulatory regions. Particularly, sunflower chitinase and lipid transfer protein (LTP), as well as fragments and variants thereof, are provided.

The compositions are useful in protecting plants from invading pathogenic organisms. One method involves stably transforming a plant with nucleotide sequences of the invention to engineer broad-spectrum disease resistance in the plant. The nucleotide sequences are expressed from a promoter capable of driving expression in a plant cell. A second method involves controlling plant pathogens by applying an effective amount of an anti-pathogenic protein or composition to the plant environment. Additionally, the nucleotide sequences of the invention are useful as genetic markers in disease-resistance breeding programs.

Promoters of the genes of the invention find use as pathogen-inducible promoters. Such promoters may be used to express other coding regions, particularly other anti-pathogenic genes, including disease and insect resistance genes.

The compositions of the invention additionally find use in agricultural and pharmaceutical compositions as antifungal and antimicrobial agents. For agricultural purposes, the compositions may be used in sprays for control of plant disease. As pharmaceutical compositions, the agents are useful as antibacterial and antimicrobial treatments.

Thus, the methods of the invention find use in controlling pests, including fungal pathogens, viruses, nematodes, insects, and the like. Transformed plants, plant cells, plant tissues, and seeds, as well as methods for making such transformed compositions are additionally provided.

### BRIEF DESCRIPTION OF THE DRAWINGS

FIG. 1 depicts the differential display of Sclerotinia-induced cDNA fragments that encode sunflower chitinase and LTP. Differential display was performed as described in Example 1. “U” indicates RNA from uninfected sunflower leaves; “I” indicates RNA from Sclerotinia-infected sunflower leaves.

FIG. 2 depicts a Northern blot analysis probed with a chitinase probe and shows the levels of chitinase transcripts in uninfected control and Sclerotinia-infected sunflower tissues. The tissues and treatments are indicated in the figure. “Oxox” tissue is from “oxox” transgenic sunflower plants expressing a wheat oxalate oxidase gene.

FIG. 3 depicts a Northern blot analysis probed with an LTP probe and shows the levels of LTP transcripts in uninfected control and Sclerotinia-infected sunflower tissues. The tissues and treatments are indicated in the figure. “Oxox” tissue is from “oxox” transgenic sunflower plants expressing a wheat oxalate oxidase gene.

FIG. 4 depicts the sequence of the chitinase promoter. Identified conserved regions, further discussed in the text, are indicated.

FIG. 5 depicts the sequence of the LTP promoter. Identified conserved regions, further discussed in the text, are indicated.

### DETAILED DESCRIPTION OF THE INVENTION

A number of terms used herein are defined and clarified in the following section.

### Definitions

By “agronomic trait” is intended a phenotypic trait of an agricultural plant that contributes to the performance or economic value of the plant. Such traits include disease resistance, insect resistance, nematode resistance, virus resistance, drought tolerance, high salinity tolerance, yield, plant height, days to maturity, seed nitrogen content, seed oil content, seed or fruit color, seed or fruit size, and the like.

By “anti-pathogenic compositions” is intended that the compositions of the invention have anti-pathogenic activity and thus are capable of suppressing, controlling, and/or killing the invading pathogenic organism. Such anti-pathogenic compositions of the invention include isolated sunflower chitinase and LTP genes and the proteins encoded thereby, as well as nucleotide and amino acid sequence fragments and variants thereof that retain their biological or regulatory function. The compositions find use in protecting plants against fungal pathogens, viruses, nematodes, insects, and the like by way of enhancing plant disease resistance. Additionally, the compositions can be used in formulations for their antibacterial and antimicrobial activities.

By “antisense DNA nucleotide sequence” is intended a sequence that is complementary to at least a portion of the messenger RNA (mRNA) for a targeted gene sequence.

By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened.

By “antipathogenic compositions” is intended that the compositions of the invention have antipathogenic activity and thus are capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

By “foreign” is intended that the transcriptional initiation region is not found in the native plant into which the transcriptional initiation region is introduced. As used herein, a chimeric gene comprises a coding sequence operably linked to a transcription initiation region that is heterogolous to the coding sequence.

By “fragment” is intended a portion of the nucleotide sequence or a portion of the amino acid sequence, and hence protein encoded thereby. Fragments of a nucleotide sequence may encode protein fragments that retain the anti-pathogenic biological activity of the native protein, and hence provide disease resistance. Alternatively, fragments of a nucleotide sequence that are useful as hybridization probes, such as described elsewhere herein, generally do not encode protein fragments that retain this biological activity. Fragments of a regulatory sequence, i.e., promoter, disclosed herein may retain their promoter activity.

By “inducible promoter” is intended that the promoter initiates expression of a gene in the presence of a pathogen, chemical, or other stimulus. Similarly, by “inducible expression” is intended that transcription of the coding sequence and subsequent translation of the messenger RNA are initiated in response to the presence of a pathogen, chemical, or other stimulus to produce an anti-pathogenic protein.

When using an inducible promoter, expression of the nucleotide sequence is initiated in cells in response to a stimulus. By “stimulus” is intended a chemical, which may be applied externally or may accumulate in response to another external stimulus; a pathogen, which may, for example, induce expression as a result of invading a plant cell; or other factor such as environmental stresses, including but not limited to, drought, temperature, and salinity.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein.

When the protein of the invention or biologically active portion thereof is recombinantly produced, preferably culture medium represents less than about 30%, 20%, 10%, or 5% (by dry weight) of chemical precursors or non-protein-of-interest chemicals.

By “nucleic acid molecule” is intended a molecule composed of nucleotides covalently bound to one another. Nucleotides include both ribonucleotides and deoxyribonucleotides. “Nucleic acid molecule” encompasses single-stranded and double stranded forms of both DNA and RNA. Nucleic acid molecules may be naturally occurring, synthetic, or a combination of both. The linear arrangement of nucleotides in a nucleic acid molecule is referred to as a “nucleotide sequence” and, unless specified otherwise, is presented herein from left to right corresponding to the 5′-to-3′ direction.

By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

By “pathogenic agent” or “pathogen” is intended any organism that has the potential to negatively impact a plant, typically, but not exclusively, by causing disease or inflicting physical damage. Such organisms include, but are not limited to, fungi, bacteria, nematodes, mycoplasmas, viruses, and insects.

By “promoter” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate.

By “stably transformed” is intended that the nucleotide sequences introduced into a cell and/or plant using transformation methods described herein are stably incorporated into the genome of the cell and/or plant. Stably incorporated nucleotide sequences are heritable.

By “variants” is intended substantially similar sequences. For nucleotide sequences, conservative variants include those sequences that, because of the degeneracy of the genetic code, encode the amino acid sequence of one of the anti-pathogenic proteins (chitinase or LTP) of the invention. Naturally-occurring allelic variants such as these can be identified with the use of well-known molecular biology techniques, as, for example, with polymerase chain reaction (PCR) and hybridization techniques as outlined below. Variant nucleotide sequences also include synthetically-derived nucleotide sequences, such as those generated, for example, by using site-directed mutagenesis or DNA shuffling as described elsewhere herein, but which still encode an anti-pathogenic protein of the invention, or, in the case of variants of a promoter sequence, retain promoter activity. Generally, variants of a particular nucleotide sequence of the invention will have at least 40%, 45%, 50%, 55%, 60%, 65%, 70%, generally at least 75%, 80%, 85%, preferably about 90% to 95% or more, and more preferably about 96%, 97%, 98%, 99% or more sequence identity to that particular nucleotide sequence as determined by sequence alignment programs described elsewhere herein using default parameters.

By “variant protein” is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention will continue to possess the desired biological activity of the native protein. Such biological activity may be, for example, anti-pathogenic activity as described herein. Such variants may result from, for example, genetic polymorphism or from human manipulation. Biologically active variants of a native anti-pathogenic protein of the invention will have at least 40%, 50%, 60%, 70%, generally at least 75%, 80%, 85%, preferably about 90% to 95% or more, and more preferably about 98% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

By “stringent conditions” or “stringent hybridization conditions” is intended conditions under which a probe will hybridize to its target sequence to a detectably greater degree than to other sequences (e.g., at least two-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC =3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C. Duration of hybridization is generally less than about 24 hours, usually about 4 to 12 hours.

Specificity is typically the function of post-hybridization washes, the critical factors being the ionic strength and temperature of the final wash solution. For DNA-DNA hybrids, the Tₘcan be approximated from the equation of Meinkoth and Wahl (1984) Anal. Biochem. 138:267-284: Tₘ=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘis the temperature (under defined ionic strength and pH) at which 50% of a complementary target sequence hybridizes to a perfectly matched probe. Tₘis reduced by about 1EC for each 1% of mismatching; thus, Tₘ, hybridization, and/or wash conditions can be adjusted to hybridize to sequences of the desired identity. For example, if sequences with ≧90% identity are sought, the Tₘcan be decreased 10EC. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘof less than 45° C. (aqueous solution) or 32° C. (formamide solution), it is preferred to increase the SSC concentration so that a higher temperature can be used. An extensive guide to the hybridization of nucleic acids is found in Tijssen (1993), Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981), Adv. Appl. Math. 2:482; the homology alignment algorithm of Needleman and Wunsch, (1970) J. Mol. Biol. 48:443-453; the search-for-similarity-method of Pearson and Lipman (1988), Proc. Natl. Acad. Sci. 85:2444-2448; the algorithm of Karlin and Altschul (1990), Proc. Natl. Acad. Sci. USA 872: 264, modified as in Karlin and Altschul (1993), Proc. Natl. Acad. Sci. USA 90:5873-5877.

Computer implementations of these mathematical algorithms can be utilized for comparison of sequences to determine sequence identity. Such implementations include but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP Version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

GAP uses the algorithm of Needleman and Wunsch (1970), J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989), Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 75% or 80%, more preferably at least 85% or 90%, and most preferably at least 95%, 96%, 97%, 98%, 99%, or 100%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 75%, 80%, 85%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 65%, 70% or 75% sequence identity to a reference sequence, preferably 80%, more preferably 85%, most preferably at least 90%, 91%, 92%, 93%, 94% or 95%; or 96%, 97%, 98%, 99%, or 100% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

### Introduction

In many plant-pathogen interactions, resistance to pathogen attack is associated with the synthesis and accumulation of proteins. Such proteins include pathogenesis-related (“PR”) proteins and other proteins, such as chitinases and lipid transfer proteins (LTPs).

Plant chitinases attack pathogens directly by degrading chitin, a component of fungal cell walls, and in this way can provide resistance to pathogen infection. Bishop et al. (2000), Proc. Natl. Acad. Sci. U.S.A. 97(10): 5322-27. Various types of chitinases have been identified in plants and categorized into several groups based on their sequences and domains. Generally, the major groups of chitinases include basic or “class I” chitinases and acidic or “class II” chitinases. Ohme-Takagi et al. (1998), Mol. Gen. Genet. 259(5): 511-15. However, the degree of structural and sequence identity between reported chitinases is low. For example, chitinases cloned from Brassica juncea (“BjCHI1”) and Nicotiana tabacum (“ChiA1”) contain chitin-binding domains, but these domains share a relatively low degree (62%) of amino acid identity. Zhao and Chye (1999), Plant Mol. Biol. 40(6): 1009-18. Further, while one chitin-binding domain is present in the Brassicajuncea chitinase BjCHI1, two are present in the Nicotiana tabacum acidic chitinase ChiA1. Similarly, although BjCHI1, which has one chitin-binding domain, therefore structurally resembles Urtica dioica agglutinin precursor UDA1, these proteins share only about 37% amino acid identity. Zhao and Chye (1999), Plant Mol. Biol. 40(6): 1009-18.

Like chitinases, lipid transfer proteins (“LTPs”) are induced in response to pathogen attack. For example, Brassica napus lipid transfer protein (“Bnltp”) is stimulated by viral infection. Sohal et al. (1999), Plant Mol. Biol. 41(1): 75-87. Generally, it is thought that plant nonspecific LTPs (nsLTPs) contain two lipid-binding sites, which may differ in their affinities for various lipids. Chavolin et al. (1999), Eur. J. Biochem. 264(2): 562-8. Because different LTPs are expressed not only in response to pathogen attack, LTPs are thought to play other roles in plant biology. Some LTPs may play a role in constitutive pathogen resistance; for example, a putative LTP from Picea abies (“Pal 8”) is constitutively expressed in embryogenic cultures and has antimicrobial activity. Sabala et al. (2000), Plant Mol. Biol. 42(3): 461-78. Other LTP expression patterns suggest other roles. For example, expression of Bnltp in epidermis of leaf and stem is consistent with the hypothesized role of LTPs in the deposition of cuticular or epicuticular waxes. Sohal et al. (1999), Plant Mol. Biol. 41(1): 75-87. Other LTPs have been shown to have different expression patterns; for example, Phaseolus vulgaris has a root-specific ns-LTP which is expressed in cortical tissue. Song et al. (1998), Plant Mol. Biol. 38(5): 735-42. Barley has an aleurone-specific gene that encodes a putative LTP; the promoter of this gene confers aleurone cell-specific expression in transgenic rice. Kalla et al. (1994), Plant J. 6(6): 849-60. The Brassica napus LTP also has different expression patterns, being expressed in lateral root initials, anthers, stigmas and vascular tissues and its stimulation by light. Sohal et al. (1999), Plant Mol. Biol. 41(1): 75-87. This has been suggested to be indicative of other functions for LTPs. Sohal et al. (1999), Plant Mol. Biol. 41(1): 75-87.

Many plants contain more than one lipid transfer protein (“LTP”), which may help to explain the variety of functions suggested for LTPs. Thoma et al. (1994), Plant Physiol. 105(1): 35-45. In sunflowers, LTP has been reported to be a cytosolic protein which can facilitate intermembrane movements in vitro. LTPs are thought to play an active role in fatty acid metabolism, which involves movements of oleyl-CoA between intracellular membranes. Arondel et al (1990), Mol. Cell. Biochem. 98(1-2): 49-56. LTPs are also thought to be involved in some aspect of secretion or deposition of lipophilic substances in cell walls, such as the cell walls of expanding epidermal cells and certain secretory tissues. Thoma et al. (1994), Plant Physiol. 105(1): 35-45.

Promoters of genes that are induced in response to pathogen attack may prove useful in regulating gene expression in an inducible manner. For example, the tomato PR gene that encodes endochitinase contains a “PR box” in its promoter region. Transcripts of this gene accumulate rapidly following an incompatible pathogen-plant interaction in tomato, and this regulation is thought to occur via a pathway involving the PR-box. Jia and Martin (1999), Plant Mol. Biol. 40(3): 455-65. These and other elements in pathogen-induced promoters may also be useful in directing expression in other ways. For example, elements in promoters may confer on operably-linked genes not only pathogen-inducibility but also tissue-preferred and/or developmentally-limited expression. By way of illustration, fragments of the potato SK2 gene promoter were fused to the reporter gene GUS; potato plants transformed with these constructs exhibited pistil-preferred and developmentally regulated expression of GUS activity. Thus, fragments of the potato SK2 promoter can be used to direct expression in a developmentally-regulated and tissue-specific manner as well as a pathogen-inducible manner. Ficker et al. (1997), Plant Mol. Biol. 35(4): 425-31. A similar means of control may be provided by the Arabidopsis thaliana class IV chitinase gene, which is constitutively expressed in seedpods of healthy plants but not in roots, inflorescence stems, leaves, and flowers. Transcripts of this gene accumulate rapidly in leaves after inoculation with Xanthomonas campestris. de A Gerhardt, et al. (1997), FEBS Lett. 419(1): 69-75. Elements responsible for such control have been identified and isolated. For example, an elicitor-responsive element (“ElRE”) has been identified in the promoter of a tobacco class I chitinase gene and used to direct expression of a reporter gene. Although transcriptional control from different elements may be a result of different mechanisms, it has been shown that regulation from this ElRE element is achieved by the binding of nuclear factors to the element. Fukuda (1997), Plant Mol. Biol. 34(1): 81-87.

### Nucleotide and Amino Acid Sequences

Compositions and methods for controlling pathogenic agents are provided. The compositions comprise two sunflower genes, including their promoters, and the anti-pathogenic proteins encoded by these genes. Methods of the invention utilize these anti-pathogenic compositions to protect plants against fungal pathogens, viruses, nematodes, insects, and the like. Additionally, the compositions can be used in formulations for their antibacterial and antimicrobial activities.

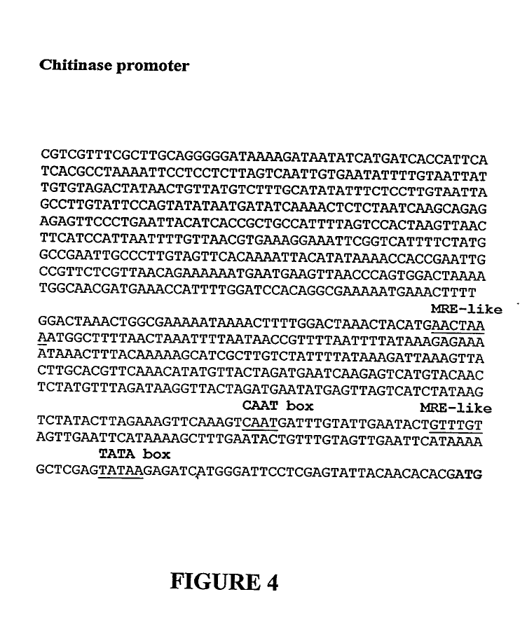

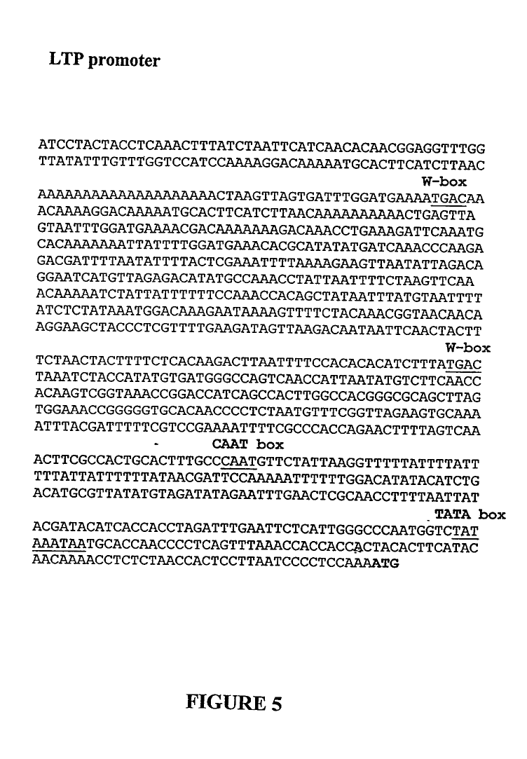

In particular, the present invention provides for isolated nucleic acid molecules comprising the nucleotide sequences set forth in SEQ ID NOs:1 or 3, the nucleotide sequences encoding the amino acid sequences set forth in SEQ ID NOs:2 or 4, the nucleotide sequences for the plant promoters set forth in SEQ ID NOs:5 or 6 and also in FIG. 4 or 5, or the nucleotide sequences encoding the DNA sequences deposited in a bacterial host as Patent Deposit No. PTA-2182. Further provided are polypeptides having an amino acid sequence set forth in SEQ ID NOs:2 or 4 and those encoded by a nucleic acid molecule described herein, for example those coding sequences set forth in SEQ ID NOS:1 or 3, and fragments and variants thereof.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence have anti-pathogenic activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

Plasmids containing the promoter sequences and gene nucleotide sequences of the invention were deposited with the Patent Depository of the American Type Culture Collection, Manassas, Va., on Jun. 30, 2000, and assigned Patent Deposit No. PTA-2182. This deposit will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

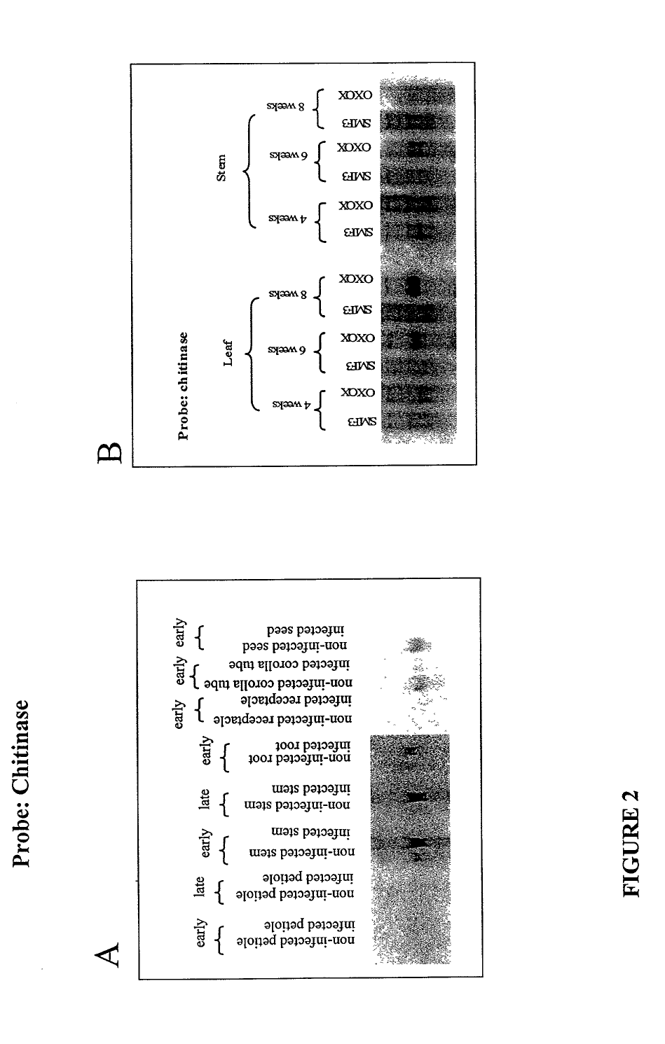

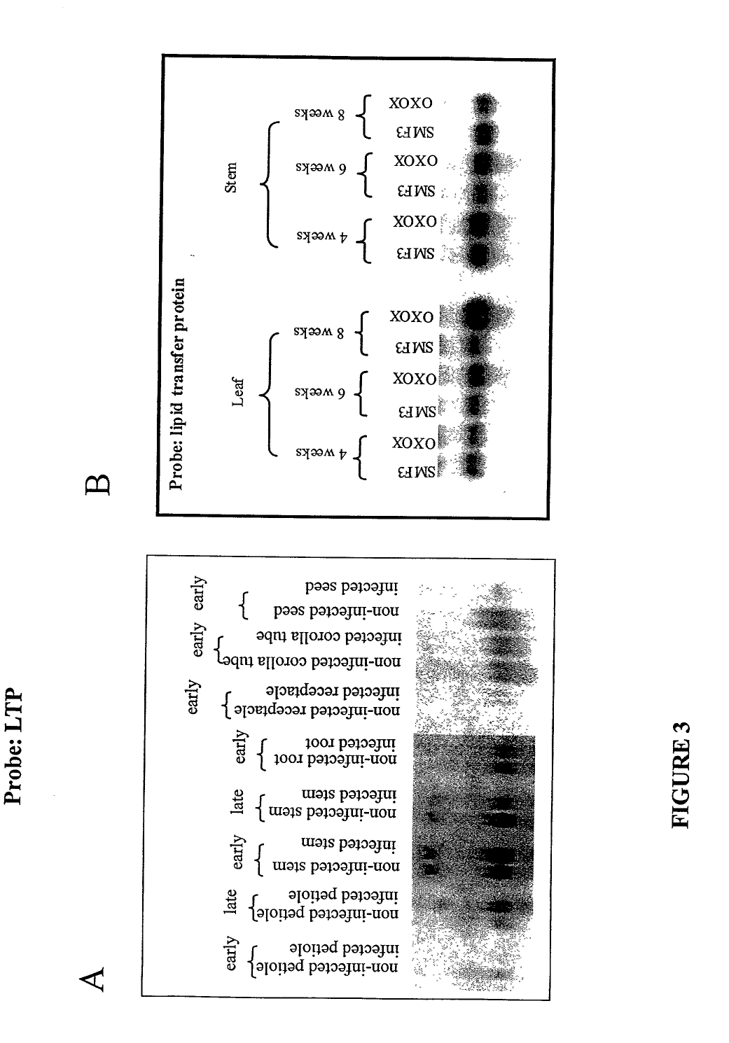

The sequences of the invention find use as anti-pathogenic agents. Thus, the genes can be used to engineer plants having disease resistance or increased disease resistance. In this manner, the sequences can be used alone or in combination with each other and/or with other known disease resistance genes to provide broad-spectrum disease resistance. For example, the chitinase and LTP gene products may prove to be useful in enhancing disease resistance in transgenic plants also expressing other transgenes. For example, oxox sunflower plants may show higher levels of chitinase and/or LTP induction in response to Sclerotinia infection, as shown in FIGS. 2 and 3.

Additionally, the sequences can be used as markers in studying defense signal pathways and in disease-resistance breeding programs. The sequences can also be used as probes to isolate other signaling components involved in defense/resistance responsiveness and to isolate the corresponding promoter sequences. See, generally, Sambrook et al. (1989) Molecular Cloning: A Laboratory Manual (2d ed., Cold Spring Harbor Laboratory Press, Plainview, N.Y.).

Compositions of the invention include the nucleotide sequences for two sunflower genes designated herein as the chitinase gene (set forth in SEQ ID NO:1) and the LTP gene (set forth in SEQ ID NO:3), and the corresponding amino acid sequences for the proteins encoded thereby (set forth in SEQ ID NO:2 and SEQ ID NO:4, respectively). Fragments and variants of these sequences as defined herein are also encompassed by the present invention. These gene sequences may be assembled into a DNA construct such that the gene is operably linked to a promoter that drives expression of a coding sequence in a cell of interest. Plants stably transformed with this DNA construct express a protein of the invention. Expression of this protein creates or enhances disease resistance in the transformed plant.

Fragments of the sunflower chitinase and LTP sequences disclosed herein are encompassed by the present invention. A fragment of a sunflower chitinase or LTP nucleotide sequence may encode a biologically active portion of a sunflower chitinase or LTP protein, or it may be a fragment that can be used as a hybridization probe or PCR primer using methods described below. A biologically active portion of a sunflower chitinase or LTP protein can be prepared by isolating a portion of one of the sunflower chitinase or LTP nucleotide sequences of the invention, expressing the encoded portion of the sunflower chitinase or LTP protein (e.g., by recombinant expression in vitro), and assessing the anti-pathogenic activity of the encoded portion of the sunflower chitinase or LTP protein. Nucleic acid molecules that are fragments of a sunflower chitinase nucleotide sequence comprise at least 16, 20, 30, 50, 60, 75, 85, 100, 125, 150, 175, 200, 225, 250, 300, 350, 400, 450, 500, 550, 600, 650, 700, 750, 800, 850, 900, 950, 1000, 1050, 1100, 1150, 1200, 1250, 1270 nucleotides, or up to the number of nucleotides present in a full-length sunflower chitinase nucleotide sequence disclosed herein (for example, 1271 nucleotides for chitinase). Nucleic acid molecules that are fragments of a sunflower LTP nucleotide sequence comprise at least 16, 20, 30, 50, 60, 75, 85, 100, 125, 150, 200, 225, 250, 300, 325, 350, 375, 400, 425, 450, 460 nucleotides, or up to the number of nucleotides present in a full-length sunflower LTP nucleotide sequence disclosed herein (for example, 460 nucleotides for LTP).

It is recognized that with these nucleotide sequences, antisense constructions complementary to at least a portion of the mRNA for the anti-pathogenic sequences can be constructed. Antisense nucleotides are constructed to hybridize with the corresponding mRNA. Modifications of the antisense sequences may be made as long as the sequences hybridize to and interfere with expression of the corresponding mRNA. Antisense constructions having 70%, preferably 80%, more preferably 85% sequence identity to the corresponding antisensed sequences may be used. Furthermore, portions of the antisense nucleotides may be used to disrupt the expression of the targeted gene. Thus, production of the native protein encoded by the targeted gene can be inhibited to achieve a desired phenotypic response. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

A fragment of the sunflower chitinase nucleotide sequence that encodes a biologically active portion of the sunflower chitinase protein of the invention will encode at least 15, 25, 30, 40, 50, 75, 100, 125, 150, 175, 200, 250, 300, 350, 370 contiguous amino acids, or up to the total number of amino acids present in a full-length chitinase protein of the invention (for example, 371 amino acid residues for chitinase). A fragment of the sunflower LTP nucleotide sequence that encodes a biologically active portion of the sunflower LTP protein of the invention will encode at least 15, 25, 30, 40, 50, 60, 70, 80, 90, 95 contiguous amino acids, or up to the total number of amino acids present in a full-length LTP protein of the invention (for example, 97 amino acid residues for LTP). Fragments of a sunflower chitinase or LTP nucleotide sequence that are useful as hybridization probes or PCR primers generally need not encode a biologically active portion of a chitinase or LTP protein.

In this manner, the present invention encompasses the anti-pathogenic proteins as well as fragments thereof. That is, it is recognized that fragments of the proteins may be produced which retain anti-pathogenic protein activity that creates or enhances disease resistance in a plant. These fragments include truncated sequences, as well as N-terminal, C-terminal, internal, and internally deleted amino acid sequences of the proteins.

The proteins of the invention may be altered in various ways including amino acid substitutions, deletions, truncations, and insertions to obtain variant proteins that continue to possess the desired anti-pathogenic activity of the native proteins disclosed herein. Methods for such manipulations are generally known in the art. For example, amino acid sequence variants of the proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. Techniques in Molecular Biology (MacMillan Publishing Company, New York) and the references cited therein. Guidance as to appropriate amino acid substitutions that do not affect desired biological activity of the native protein may be found in the model of Dayhoff et al. (1978) Atlas of Protein Sequence and Structure (Nat'l Biomed. Res. Found., Washington, D.C.), herein incorporated by reference. Conservative substitutions, such as exchanging one amino acid with another having similar properties, may be preferable.

Thus, the genes and nucleotide sequences of the invention include both the naturally occurring sequences as well as mutant forms. Likewise, the proteins of the invention encompass the naturally occurring proteins as well as variations and modified forms thereof. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequences encompassed herein are not expected to produce radical changes in the characteristics of the anti-pathogenic proteins. However, when it is difficult to predict the exact effect of the substitution, deletion, or insertion in advance of doing so, one skilled in the art will appreciate that the effect will be evaluated by routine screening assays. That is, the activity of the modified protein sequences can be evaluated by monitoring of the plant defense system in response to Sclerotinia attack. See, for example U.S. Pat. No. 5,614,395, herein incorporated by reference.

Variant nucleotide sequences and proteins also encompass anti-pathogenic genes and proteins derived from a mutagenic and recombinogenic procedure such as DNA shuffling. With such a procedure, one or more different anti-pathogenic gene or protein sequences can be manipulated to create new sequences possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the sunflower chitinase or LTP gene of the invention and other known anti-pathogenic genes to obtain a new gene encoding a protein with an improved property of interest, such as a broader spectrum of pathogen resistance. Likewise, sequences corresponding to regulatory motifs, such as specific cis-acting elements within the promoters of the invention may be shuffled creating improved regulatory functions, such as increased pathogen inducibility. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire anti-pathogenic promoters and genes of the present invention or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the sunflower chitinase or LTP sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire anti-pathogenic coding sequence or a portion thereof may be used as a probe capable of specifically hybridizing to corresponding coding sequences and messenger RNAs. To achieve specific hybridization under a variety of conditions, such probes include sequences that are unique and are preferably at least about 10 nucleotides in length, and most preferably at least about 20 nucleotides in length. Such probes may be used to amplify the anti-pathogenic coding sequences from a chosen organism by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions as qualified elsewhere herein. Isolated sequences that have anti-pathogenic activity and which hybridize under stringent conditions to the chitinase and LTP gene sequences disclosed herein, or to fragments thereof, are encompassed by the present invention.

While the invention is not bound by any particular mechanism of action, the gene products, probably proteins or polypeptides, function to inhibit or prevent plant diseases in a plant. Such gene products may be anti-pathogenic. That is, such gene products may be capable of suppressing, controlling, and/or killing the invading pathogenic organism. It is recognized that the present invention is not dependent upon a particular mechanism of defense. Rather, the genes and methods of the invention work to increase resistance of the plant to pathogens independently of how that resistance is accomplished.

The anti-pathogenic genes and proteins of the invention, as well as fragments and variants thereof, can also be used to control resistance to pathogens by creating or enhancing defense mechanisms in a plant. While the exact function of the anti-pathogenic proteins is not known, these proteins are involved in influencing the expression of defense-related proteins. It is recognized that the present invention is not premised upon any particular mechanism of action of the anti-pathogenic genes. It is sufficient for purposes of the invention that the genes and proteins are involved in the plant defense system and can be used to create or increase resistance levels in the plant to pathogens.

The plant defense mechanisms described herein may be used alone or in combination with other proteins or agents to protect against plant diseases and pathogens. Other plant defense proteins include those described in the copending application entitled “Methods for Enhancing Disease Resistance in Plants,” U.S. application Ser. No. 09/256,898, filed Feb. 24, 1999, the copending application entitled “Genes for Activation of Plant Pathogen Defense Systems,” U.S. application Ser. No. 09/256,158, filed Feb. 24, 1999, and the copending application entitled “Family of Maize PR-1 Genes and Promoters,” U.S. application Ser. No. 09/257,583, filed Feb. 25, 1999, all of which are herein incorporated by reference.

The anti-pathogenic nucleotide sequences of the invention are provided in expression cassettes for expression in the plant of interest as described below. The cassette will include 5′ and 3′ regulatory sequences operably linked to an anti-pathogenic sequence of the invention.

A number of promoters can be used to drive the expression of the coding sequences encoding the anti-pathogenic proteins of the invention. The promoters may be selected based on the desired outcome. For example, the promoters may be selected based on desired timing, localization, and/or level of expression of the anti-pathogenic genes in a plant. Constitutive, tissue-preferred, pathogen-inducible, and wound-inducible promoters can be used in the practice of the invention. The promoter used to regulate expression of the claimed nucleotide sequence may be homologous to the claimed nucleotide sequence. In these cases, the transformed plant will have a change in phenotype. The anti-pathogenic coding sequences of the invention may be expressed by promoters that are native or analogous or foreign or heterologous to the operably linked coding sequence. A number of heterologous promoters can be used toward this end.

It may be beneficial to express the gene from an inducible promoter, particularly a pathogen-inducible promoter. The inducible promoter will initiate expression of a gene in the presence of a pathogen to prevent infection and disease symptoms. Such promoters include those from other pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al (1992) Plant Cell 4:645-656; and Van Loon (1985) Plant Mol. Virol. 4:111-116. See, also the copending application entitled “Maize PR-1 Genes and Promoters”, U.S. application Ser. No. 09/257,583, filed Feb. 25, 1999, herein incorporated by reference.

Of interest are promoters that are expressed locally at or near the site of pathogen infection. See, for example, Marineau et al. (1987) Plant Mol. Biol. 1:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al (1996) Plant J. 10:955-966; Zhang et al. (1994) Proc. Natl. Acad. Sci. USA 91:2507-2511; Warner et al. (1993) Plant J. 3:191-201; Siebertz et al (1989) Plant Cell:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, the copending application entitled “Constitutive Maize Promoters,”U.S. application Ser. No. 09/257,584, filed Feb. 25, 1999, and herein incorporated by reference.

Tissue-preferred promoters can be used to target anti-pathogenic gene expression within a particular tissue. Tissue-preferred promoters include Yamamoto et al. (1997) Plant J. 12(2)255-265; Kawamata et al. (1997) Plant Cell Physiol. 38(7):792-803; Hansen et al (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase); and ce1A (cellulose synthase) (see the copending application entitled “Seed-Preferred Promoters,” U.S. application Ser. No. 09/377,648, filed Aug. 19, 1999, herein incorporated by reference). Gama-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2): 207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):l 1′-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed ro1C and ro1D root-inducing genes of Agrobacterium rhizogenes (see Plant Science (Limerick) 79(1):69-76). They concluded that enhancer and tissue-specific DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and ro1B promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

In one embodiment, the nucleic acids of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999). See also Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

### Sunflower Promoters

The invention also encompasses the 5′ regulatory regions of the chitinase (SEQ ID NO:5) and lipid transfer protein (LTP; SEQ ID NO:6) genes disclosed herein. The nucleotide sequences for the native 5′ untranslated regions, i.e., promoters, are provided in SEQ ID NO:5 and SEQ ID NO:6, respectively. It is recognized that, having identified the nucleotide sequences for the promoter regions disclosed herein, it is within the state of the art to isolate and identify further regulatory elements in the 5′ untranslated region upstream from the particular promoter regions identified herein. Thus, for example, the promoter regions disclosed herein may further comprise upstream regulatory elements that confer tissue-specific and/or tissue-preferred expression of any heterologous nucleotide sequence operably linked to one of the disclosed promoter sequences. See particularly Australian Patent No. AU-A-77751/94 and U.S. Pat. Nos. 5,466,785 and 5,635,618. Likewise, promoter regions having homology to the promoters of the invention can be isolated by hybridization under stringent conditions, as described elsewhere herein.

Pathogen-responsive cis-acting elements have been identified within these promoter regions, such as MRE-like elements, a TATA-box-like element, and a CAAT-box-like element in the chitinase promoter (shown in FIG. 4), and a TATA-box-like element and CAAT-box-like element and pathogen-responsive elements such as W-Box-like elements in the LTP promoter (shown in FIG. 5). These promoters have been identified as having an inducible expression pattern. Thus, where gene expression in response to a stimulus is desired, an inducible promoter of the invention is the regulatory element of choice. When using an inducible promoter, expression of the nucleotide sequence is initiated in cells in response to a stimulus, as described elsewhere herein.

The promoter sequences of the invention include both the naturally occurring sequences as well as mutant forms. Additionally, sequences corresponding to regulatory motifs, such as specific cis-acting elements within the promoters of the invention may be shuffled to create improved regulatory functions such as increased pathogen inducibility. Strategies for such DNA shuffling are known in the art. See, for example, Stemmer (1994), Proc. Natl. Acad. Sci. USA 91:10747-10751; Stemmer (1994), Nature 370:389-391; Crameri et al. (1997), Nature Biotechnology 15:436-438; Moore et al. (1997), J. Mol. Biol. 272:336-347; Zhang et al. (1997), Proc. Natl. Acad. Sci. USA 94:4504-4509; Crameri et al. (1998), Nature 391:288-291; and U.S. Pat. Nos. 5,605,793 and 5,837,458.

Fragments and variants of the promoter nucleotide sequences disclosed herein are also encompassed by the present invention. A fragment of a sunflower chitinase promoter nucleotide sequence comprises at least 16, 20, 30, 50, 60, 75, 85, 100, 125, 150, 175, 200, 225, 250, 300, 350, 400, 450, 500, 550, 600, 650, 700, 750, 800 nucleotides, or up to the number of nucleotides present in a full-length sunflower chitinase promoter nucleotide sequence disclosed herein (for example, 850 nucleotides for chitinase promoter). Nucleic acid molecules that are fragments of a sunflower LTP promoter nucleotide sequence comprise at least 16, 20, 30, 50, 60, 75, 85, 100, 125, 150, 175, 200, 225, 250, 300, 350, 400, 450, 500, 550, 600, 650, 700, 750, 800, 850, 900, 950, 1000, nucleotides, or up to the number of nucleotides present in a full-length sunflower LTP promoter nucleotide sequence disclosed herein (for example, 1040 nucleotides for LTP promoter).

Generally, fragments of a promoter sequence that retain their biological activity comprise at least 30, 35, or 40 contiguous nucleotides, preferably at least 50 contiguous nucleotides, more preferably at least 75 contiguous nucleotides, still more preferably at least 100 contiguous nucleotides of the particular promoter nucleotide sequence disclosed herein. Preferred fragment lengths depend upon the objective and will also vary depending upon the particular promoter sequence.

The nucleotides of such fragments will usually comprise the TATA recognition sequence of the particular promoter sequence. Such fragments may be obtained by use of restriction enzymes to cleave the naturally occurring promoter nucleotide sequence disclosed herein; by synthesizing a nucleotide sequence from the naturally occurring sequence of the promoter DNA sequence; or may be obtained through the use of PCR technology. See particularly, Mullis et al. (1987) Methods Enzymol. 155:335-350, and Erlich, ed. (1989) PCR Technology (Stockton Press, New York). Variants of these promoter fragments, such as those resulting from site-directed mutagenesis, are encompassed by the compositions of the present invention.

The nucleotide sequences for the inducible promoters disclosed in the present invention, as well as variants and fragments thereof, are useful in the genetic manipulation of any plant when assembled within a DNA construct such that the promoter sequence is operably linked with a heterologous nucleotide sequence whose inducible expression is to be controlled to achieve a desired phenotypic response. It is recognized that the promoter sequences of the invention may also be used with their native coding sequences to increase or decrease expression of the native coding sequence, thereby resulting in a change in phenotype in the transformed plant.

The promoters of the invention can be used to regulate expression of any nucleotide sequence of interest in order to vary the phenotype of a plant. Such expression may be regulated by the promoters of the invention in an inducible manner. Various changes in phenotype are of interest. Nucleotide sequences of interest include, for example, disease resistance genes, insect resistance genes, and the like. Other sequences of interest include antisense nucleotide sequences.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; Geiser et al. (1986) Gene 48:109); lectins (Van Damme et al. (1994) Plant Mol. Biol. 24:825); protease inhibitors (Ryan et al. (1990) Ann. Rev. Phytopathol, 28:425-449); tachyplesin (U.S. patent application Ser. No. 08/962,034); amylase inhibitors (Fung et al. (1996) Insect Biochem. Mol. Biol. 26(5):419-426, and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; Mindrinos et al. (1994) Cell 78:1089); and the like.

### Expression Cassettes

The nucleotide sequences of the invention are provided in expression cassettes for use in the plant of interest. Expression cassettes may comprise any of the nucleotide sequences of the invention. For example, expression cassettes or DNA constructs of the invention may be provided with a plurality of restriction sites for insertion of the anti-pathogenic sequence to be under the transcriptional regulation of the regulatory regions. Expression cassettes or DNA constructs may also be provided with a plurality of restriction sites for insertion of a sequence of interest to be placed under the regulatory influence of the promoters of the invention. The expression cassettes may additionally at least one additional gene to be cotransformed into the organism. Alternatively, the additional gene(s) can be provided on multiple expression cassettes. The expression cassette may additionally contain selectable marker genes.

The expression cassettes or DNA constructs of the invention will include in the 5′-to-3′ direction of transcription, a transcriptional and translational initiation region, a nucleotide sequence to be expressed, and a transcriptional and translational termination region functional in plants. The transcriptional initiation region, the promoter, may be native or analogous or foreign or heterologous to the plant host. The promoter may also be native or analogous or foreign or heterologous to the nucleotide sequence or coding sequence to be expressed. Additionally, the promoter may be the natural sequence or alternatively a synthetic sequence.

While it may be preferable to express the sequences encoding the anti-pathogenic proteins using heterologous promoters, the native promoter sequences may be used. Such constructs would change expression levels of the anti-pathogenic genes in the plant or plant cell. Thus, the phenotype of the plant or plant cell is altered.

The termination region may be native with respect to the transcriptional initiation region, may be native with respect to the operably linked DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al (1990) Gene 91:151-158; Ballas et al. 1989) Nucleic Acids Res. 17:7891-7903; Joshi et al. (1987) Nucleic Acids Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831 and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp.177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724; etc. Such disclosures are herein incorporated by reference. The above list of selectable marker genes is not meant to be limiting. Any selectable marker gene can be used in the present invention.

It is further recognized that the components of the expression cassettes may be modified to increase expression. For example, truncated sequences, nucleotide substitutions, or other modifications may be employed. See, for example Perlak et al. (1991) Proc. Natl. Acad. Sci. USA 88:3324-3328; Murray et al. (1989) Nucleic Acids Res. 17:477-498; and WO 91/16432.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86: 6126-6130); potyvirus leaders, for example, TEV leader (Tobacco Etch Virus) (Gallie et al. (1995) Gene 165(2): 233-238), MDMV leader (Maize Dwarf Mosaic Virus) (Virology 154: 9-20), and human immunoglobulin heavy-chain binding protein (BiP) (Macejak et al. (1991) Nature 353: 90-94; untranslated leader from the coat protein mRNA of alfalfa mosaic virus (AMV RNA 4) (Jobling et al. (1987) Nature 325: 622-625); tobacco mosaic virus leader (TMV) (Gallie. (1989) Molecular Biology of RNA, ed. Cech (Liss, N.Y.) pp. 237-256; and maize chlorotic mottle virus leader (MCMV) (Lommel et al. (1991) Virology 81:382-385). See also, Della-Cioppa et al (1987) Plant Physiology 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

### Transformation

The genes and promoters of the present invention can be used to transform any plant. In this manner, genetically modified plants, plant cells, plant tissue, seed, and the like can be obtained. Transformation protocols may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromnn et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Pnaicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g. Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

### Virus-Mediated Transformation

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that a chitinase or LTP polypeptide of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

### Pathogens and Pests

The invention is drawn to compositions and methods for inducing resistance in a plant to plant pests. Accordingly, the compositions and methods are also useful in protecting plants against fungal pathogens, viruses, nematodes, insects, and the like.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphamidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphamidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphamidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphamidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphamidermatum, Aspergillusflavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium monilforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera and Globodera spp; particularly Globodera rostochiensis and globodera pailida (potato cyst nematodes); Heterodera glycines (soybean cyst nematode); Heterodera schachtii (beet cyst nematode); and Heterodera avenae (cereal cyst nematode).

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworn; Helicoverpa zea, cotton bollworm; Spodoptera exigua, beet armyworm; Pectinophora gossypiella, pink bollworm; Anthonomus grandis grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

### Molecular Markers

The present invention provides a method of genotyping a plant comprising a polynucleotide of the present invention. The plant may be a monocot, such as maize or sorghum, or alternatively, a dicot, such as sunflower or soybean. Genotyping provides a means of distinguishing homologs of a chromosome pair and can be used to differentiate segregants in a plant population. Molecular marker methods are useful for a variety of purposes, such as phylogenetic studies, characterizing genetic relationships among crop varieties, identifying crosses or somatic hybrids, localizing chromosomal segments affecting monogenic traits, map-based cloning, and the study of quantitative inheritance. See, e.g., Clark, ed. (1997) Plant Molecular Biology: A Laboratory Manual, Chapter 7 (Springer-Verlag, Berlin). For molecular marker methods, see generally, Paterson (1996) “The DNA Revolution,” in Genome Mapping in Plants, ed. Paterson (Academic Press/R. G. Landis Company, Austin, Tex.), pp. 7-21.

The particular method of genotyping in the present invention may employ any number of molecular marker analytic techniques such as, but not limited to, restriction fragment length polymorphisms (RFLPs). Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, preferably within 20 or 10 cM, more preferably within 5, 3, 2, or 1 cM of a gene of the invention.

In the present invention, the nucleic acid probes employed for molecular marker mapping of plant nuclear genomes selectively hybridize, under selective hybridization conditions, to a gene encoding a polynucleotide of the present invention. In some embodiments, the probes are selected from polynucleotides of the present invention. Typically, these probes are cDNA probes or Pst I genomic clones. The length of the probes is discussed in greater detail, supra, but is typically at least 15 bases in length, more preferably at least 20, 25, 30, 35, 40, or 50 bases in length. Generally, however, the probes are less than about 1 kilobase in length. Preferably, the probes are single copy probes that hybridize to a unique locus in a haploid chromosome complement.

The present invention further provides a method of genotyping comprising the steps of contacting, under stringent hybridization conditions, a sample suspected of comprising a polynucleotide of the present invention with a nucleic acid probe. Generally, the sample is a plant sample, preferably, a sample suspected of comprising a sunflower polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In some embodiments, the nucleic acid probe comprises a polynucleotide of the present invention.

### Formulations

Methods are provided for controlling plant pathogens comprising applying an anti-pathogenic amount of a protein or composition of the invention to the environment of the pathogens. The proteins of the invention can be formulated with an acceptable carrier into a pesticidal composition(s) that is, for example, a suspension, a solution, an emulsion, a dusting powder, a dispersible granule, a wettable powder, an emulsifiable concentrate, an aerosol, an impregnated granule, an adjuvant, a coatable paste, and also encapsulations in, for example, polymer substances. Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors or other preparations that influence plant growth. One or more agrochemicals including, but not limited to, herbicides, insecticides, fungicides, bacteriocides, nematocides, molluscicides, acaracides, plant growth regulators, harvest aids, and fertilizers, can be combined with carriers, surfactants, or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present invention are normally applied in the form of compositions and can be applied to the crop area or plant to be treated, simultaneously or in succession, with other compounds. In some embodiments, methods of applying an active ingredient of the present invention or an agrochemical composition of the present invention (which contains at least one of the proteins of the present invention) are foliar application, seed coating, and soil application. The number of applications and the rate of application depend on the intensity of infestation by the corresponding pest.

Suitable surface-active agents include, but are not limited to, anionic compounds such as a carboxylate of, for example, a metal; a carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g. polyoxyethylene sorbitar fatty acid esters, block copolymers of ethylene oxide and propylene oxide, acetylenic glycols such as 2, 4, 7, 9-tetraethyl-5-decyn-4,7-diol, or ethoxylated acetylenic glycols. Examples of a cationic surface-active agent include, for instance, an aliphatic mono-, di-, or polyamine such as an acetate, naphthenate, or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions of the present invention can be in a suitable form for direct application or as concentrate of primary composition, which requires dilution with a suitable quantity of water or other diluent before application. The pesticidal concentration will vary depending upon the nature of the particular formulation, specifically, whether it is a concentrate or to be used directly. The composition contains 1 to 98% of a solid or liquid inert carrier, and 0 to 50%, preferably 0.1 to 50% of a surfactant. These compositions will be administered at the labeled rate for the commercial product, preferably about 0.01 lb-5.0 lb per acre when in dry form and at about 0.01 pts-10 pts per acre when in liquid form.

In a further embodiment, the compositions, as well as the proteins of the present invention can be treated prior to formulation to prolong the activity when applied to the environment of a target pest as long as the pretreatment is not deleterious to the activity. Such treatment can be by chemical and/or physical means as long as the treatment does not deleteriously affect the properties of the composition(s). Examples of chemical reagents include, but are not limited to, halogenating agents; aldehydes such as formaldehyde and glutaraldehyde; anti-infectives, such as zephiran chloride; alcohols, such as isopropanol and ethanol; and histological fixatives, such as Bouin=s fixative and Helly=s fixative (see, for example, Humason (1967) Animal Tissue Techniques (W.H. Freeman and Co.).

The compositions can be applied to the environment of a pest by, for example, spraying, atomizing, dusting, scattering, coating or pouring, introducing into or on the soil, introducing into irrigation water, by seed treatment, or dusting at the time when the pest has begun to appear or before the appearance of pests as a protective measure. It is generally important to obtain good control of pests in the early stages of plant growth, as this is the time when the plant can be most severely damaged. The compositions of the invention can conveniently contain another insecticide or pesticide if this is thought necessary.

In a further embodiment, formulations of the present invention for use as antimicrobial therapies comprise the anti-pathogenic proteins in a physiologically or pharmaceutically acceptable carrier, such as an aqueous carrier. Thus, formulations for use in the present invention include, but are not limited to, those suitable for parenteral administration, including subcutaneous, intradermal, intramuscular, intravenous and intraarterial administration, as well as topical administration. The formulations may conveniently be presented in unit dosage form and may be prepared by any of the methods well known in the art. Such formulations are described in, for example, Remington's Pharmaceutical Sciences (19th ed., Mack Pub. Co., Easton, Pa., 1995).

In the manufacture of a medicament according to the invention, the anti-pathogenic compositions are typically admixed with, inter alia, an acceptable carrier. The carrier must, of course, be acceptable in the sense of being compatible with any other ingredients in the formulation and must not be deleterious or harmful to the patient. The carrier may be a solid or a liquid. One or more anti-pathogenic proteins may be incorporated in the formulations of the invention, which may be prepared by any of the well-known techniques of pharmacy consisting essentially of admixing the components, optionally including one or more accessory therapeutic ingredients.

Formulations of the present invention may comprise sterile aqueous and non-aqueous injection solutions of the active compound, which preparations are preferably isotonic with the blood of intended recipient and essentially pyrogen free. These preparations may contain anti-oxidants, buffers, bacteriostats, and solutes that render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water for injection immediately prior to use.

In the formulation, the anti-pathogenic protein may be contained within a lipid particle or vesicle, such as a liposome or microcrystal, which may be suitable for parenteral administration. The particles may be of any suitable structure, such as unilamellar or plurilamellar, so long as the targeted cassette is contained therein. Positively charged lipids such as N-[1-(2,3-dioleoyloxi)propyl]-N,N,N-trimethyl-amoniummethylsulfate, or “DOTAP”, may be used for such particles and vesicles. The preparation of such lipid particles is well known. See, e.g., U.S. Pat. Nos. 4,880,635; 4,906,477; 4,911,928; 4,917,951; 4,920,016; 4,921,757; etc.

The dosage of the anti-pathogenic protein administered will vary with the particular method of administration, the condition of the subject, the weight, age, and sex of the subject, the particular formulation, the route of administration, etc. In general, the protein will be administered in a range of about 1 μg/L to about 10 g/L.

### Chimeraplasty

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

The following examples are offered by way of illustration and not by way of limitation.

### Experimental

### EXAMPLE 1

### Isolation of the Sunflower Chitinase and LTP cDNA and Promoter Clones

Plant Material

Sunflower (Helianthus, SMF3) plants were grown in the greenhouse or growth chamber. Pathogen Sclerotinia sclerotiorum (255M⁷) was maintained on PDA plates at 20° C. in the dark.

Preparation of Total RNA

Sunflower tissues were ground in liquid nitrogen and total RNA was isolated using the Tri-Reagent Method (Sigma), according to the manufacturer's protocol.

Differential Display

Differential display was carried out according to the method developed by Liang and Pardee (1992), Science 257::967-971, using total RNA from Sclerotinia-infected and uninfected sunflower leaf tissues. Three petioles per plant (six-week-old) were infected with Sclerotinia mycelia. Leaf tissues were harvested three days after inoculation. The potential Sclerotinia-induced cDNA fragments were isolated from the gel and amplified using the primers shown in Table 1 (primers AP-4 and AP-2 set forth in SEQ ID NOS:7 and 8, respectively).

TABLE 1
Primers used for isolation of cDNA fragments
in the differential display assay

| Gene | Primer | Sequence |
|---|---|---|
| chitinase | T12MC | CMAAAAA-(A)n |
| | AP-4 | 5′-GGTACTCCAC-3′ |
| LTP | T12MC | GMAAAAA-(A)n |
| | AP-2 | 5′-GACCGCTTGT-3 |

The PCR products were cloned into the TA vector (INVITROGEN) and sequenced with an ABI 373 Automated DNA sequencer. The gene-specific primers were designed based on the sequences of the cDNA fragments.

Isolation of Full-Length cDNA Clone

The full-length cDNA clones were isolated by using RACE-like PCR-based technology. The sequence information generated from the differential display was used to design gene-specific primers to amplify the 5′ end regions of the target genes using PCR-based RACE technology. Sclerotinia-infected leaf and oxalate oxidase-transgenic stem cDNA libraries (2:1 ratio) were used as template. To facilitate cloning full-length cDNAs from initial cloned regions, we designed a 28-bp vector primer flanking the cDNA on the 5′ end of the pBS vector; we then directionally amplified the 5′ ends of the cDNAs of the two genes using their respective gene-specific primers (see Table 2).

TABLE 2
Primers used for isolation of full-length cDNA clones

| GENE | ORIENTATION | SEQUENCE | cDNA | SEQ ID NO: |
|---|---|---|---|---|
| LTP | reverse | AACACAAACAAACACGTTACATCAGT | partial, 5′-end | 9 |
| | forward | TCCGGCTCGTATGTTGTGTGGAATTG | 10 | |
| chitinase | forward | CACATGTCTTTCAACTGTCACCAGGGAG | 3′-end | 11 |
| | reverse | GCGATTAAGTTGGGTAACGCCAGGGT | 12 | |
| | forward | TCCGGCTCGTATGTTGTGTGGAATTG | 5′-end | 13 |
| | reverse | CAAGCAGTCCATGTCTGCGAAGCTAGTC | 14 | |

PCR reactions were performed in a total volume of 50 μl in 10 mM Tris-HCl, pH 8.3; 1.5 mM MgCl₂; 50 mM KCl; 0.1 mM dNTPs; and 0.25 μM of each primer with 0.5 units of Advantage cDNA polymerase mix (Clontech).

Northern Blot Assay

Total RNA (20 μg) was separated in a 1% agarose gel containing formaldehyde (Sambrook et al (1989) Molecular Cloning: A Laboratory Manual (2d ed., Cold Spring Harbor Laboratory Press, Plainview N.Y.), pp. 7.43-7.52). Ethidium bromide was included to verify equal loading of RNA. After transfer onto Hybond N+ membrane, the blots were hybridized with ³²P-labeled chitinase or LTP cDNA. Hybridization and washing conditions were performed according to Church and Gilbert (1984) Proc. Natl Acad. Sci. USA 81:1991-1995.

Isolation of Promoter Regions

Promoter regions of chitinase and LTP were isolated from sunflower genomic DNA using Universal GenomeWalker Kit (Clontech) according to the manufacturer's instructions. Restriction-digested genomic DNAs were ligated with an adapter to construct pools of genomic DNA fragments for walking by PCR (Siebert et al. (1995) Nucleic Acids Res. 23:1087-1088). Gene specific primers were designed for the walking procedure (Table 3).

TABLE 3

Primers for isolation of promoter regions
of chitinase and LTP.

| | | | |
|---|---|---|---|
| GENE | FUNCTION | SEQUENCE | SEQ ID NO: |

| | | | |
|---|---|---|---|
| LTP | primary PCR primer | 5′-GTAATACGACTCACTATAGGGC-3′ | 15 |
| | secondary PCR primer | 5′-ACTATAGGGCACGCGTGGT-3′ | 16 |
| | gene-specific | 5′-CAGGGAGTTGGCCCATAAAGACCATCAT-3′ | 17 |
| chitinase | primary PCR primer | 5′-GTAATACGACTCACTATAGGGC-3′ | 18 |
| | secondary PCR primer | 5′-ACTATAGGGCACGCGTGGT-3′ | 19 |
| | gene-specific | 5′-GGGCGCTTATAGGACTACAAATGGCAAG-3′ | 20 |

DNA and Protein Sequence Analysis

DNA sequence analysis was carried out with the Sequencher (3.0). Multiple-sequence alignments (Clustal W) of the amino acid sequences were analyzed with Curatool (CuraGen).

Construction of Sunflower cDNA Libraries

Six-week-old SMF3 sunflower plants were infected with Sclerotinia sclerotrium by petiole inoculation with Sclerotinia-infected carrot plugs. Six days after infection, leaf and stem tissues were collected from infected plants for total RNA isolation. Total RNA was also isolated from sunflower oxalate oxidase-transgenic plants (line 610255) expressing a wheat oxalate oxidase gene at the six-week stage. The mRNAs were isolated using an mRNA purification kit (BRL) according to the manufacturer's instruction. cDNA libraries were constructed with the ZAP-cDNA synthesis kit into pBluescript phagemid (Stratagene). A cDNA library mixture for PCR cloning was made of Sclerotinia-infected leaf and oxox transgenic stem libraries (2:1 ratio).

Results

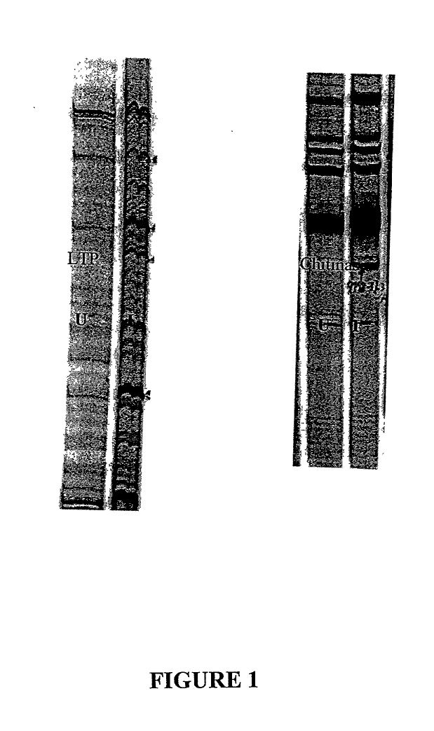

A total of 18 Sclerotinia highly induced cDNA bands were identified on the differential display sequence gel (FIG. 1). Two of the bands were isolated and further characterized.

The full-length chitinase cDNA isolated from the sunflower cDNA library of Sclerotinia-infected sunflower leaf is 1271 bp long with an open reading frame encoding a protein of 371 amino acid residues having a molecular weight of approximately 40.8 kDa and a pI of about 8.60. A GenBank database search revealed that sunflower chitinase shares homology at the amino acid level with other plant chitinases, showing about 52% similarity and about 43% identity with a chitinase from Nicotiana tabacum (GenBank Accession No. Q43591); about 50% similarity and 43% identity with a chitinase from N. tabacum (GenBank Accession No. Q43576); about 48% similarity and 42% identity with Arabidopsis thaliana chitinase (GenBank Accession No. Q81862).

The full-length LTP cDNA isolated from the sunflower cDNA library of Sclerotinia-infected sunflower leaf is 475 bp long with an open reading frame encoding a protein of 97 amino acid residues having a molecular weight of about 10.2 kDa and a pI of 8.32. A GenBank database search revealed that sunflower LTP shares homology at the amino acid level with other plant LTPs, showing about 60% similarity and about 47% identity with an LTP from Zinnia elegans (GenBank Accession No. Q42392); about 54% similarity and about 43% identity with an LTP from Senecio odorus (GenBank Accession No. Q41378); about 51% similarity and about 42% identity with Vigna unguiculata LTP (GenBank Accession No. NTLP_VIGUN); about 49% similarity and about 40% identity with Arabidopsis thaliana LTP (GenBank Accession No. Q42158); about 53% similarity and about 40% identity with an LTP from Brassica rapa (GenBank Accession No. Q64431); about 36% similarity and about 28% identity with Hordeum vulgare LTP (GenBank Accession No. Q81135); about 39% similarity and about 31% identity with an LTP from Oryza sativa (GenBank Accession No. Q40631).

The 5′-flanking sequence of the chitinase gene contains a putative TATA-box, a CAAT-box, and two putative pathogen-responsive, MRE-like elements (FIG. 4). The LTP promoter region contains a putative TATA box, a CAAT-box, and putative pathogen-responsive elements, such as a W-box (FIG. 5).

### EXAMPLE 2

### Induction of Steady-State Level of Chitinase and LTP Transcripts by Sclerotinia Infection and Chemical Treatment

Fungal Infection and Chemical Treatments

A. Middle Stock Infection.

Sunflower (SMF3 and transgenic “oxox” (described in Example 3)) plants were planted in 4-inch pots and grown in a greenhouse under standard conditions for four weeks. After transfer to a growth chamber, plants were maintained under a 12-hour photoperiod at 22° C. at 80% relative humidity. Six-week old plants were inoculated with Sclerotinia-infected carrot plugs; for each plant, three petioles were inoculated and then wrapped with a 1×2 inch section of Parafilm®.

B. Root Infection.

Selected Sclerotinia isolates were combined and homogenized in predetermined proportions. Inoculations were administered so as to best approximate actual field conditions of disease appearance on sunflowers. Thus, the inoculation was usually performed at the R3 to R4 stage of sunflower growth, which occurs one to two weeks prior to flowering (see, Sunflower Production Handbook. 1994 North Dakota State University, Fargo, N. Dak., Extension Bulletin 25). An inoculant envelope containing a premeasured amount of inoculum was prepared for each plant, and the inoculum load was delivered about 1.5 inches into the soil at a distance from the plant base of about 1.5 inches. Immediately upon completion of inoculation, the soil was lightly irrigated. Inoculated plants were then monitored for symptoms of Sclerotinia basal stalk rot, which should begin to appear approximately 2 weeks after inoculation.

C. Head Infection.

Greenhouse-grown sunflowers were inoculated with Sclerotinia ascospores at the R-5.5 stage of flower development. The ascospore inoculum was delivered in precise amounts, which may be calculated with the aid of a hemacytometer. Inoculation was accomplished by spraying sunflowers directly in the floral surface of the flower heads. Inoculated heads were then covered with non-breathable bags (such as plastic gallon-size food storage bags), which were sealed to ensure high humidity conditions. Greenhouse lights were turned off for 24 hours after inoculation to prevent drying or high temperatures that might slow or stop infection. Bags were removed after 72 hours.

Six week-old sunflower (SMF3) plants were treated with different chemicals in the greenhouse. Salicylic acid, oxalic acid and hydrogen peroxide were purchased from Sigma (St. Louis, USA), and jasmonic acid was obtained from Apex Org. (UK). For chemical treatments, plant leaves were sprayed until runoff with 5 mM SA, 5 mM of oxalic acid, 5 mM H₂O₂, and/or 45 uM JA (in 0.1% ethanol).

D. Tissue Collection and Northern Blot Assays

Plant tissue samples were collected at different time points by immediately freezing in liquid nitrogen and were then stored at −80° C. Total RNA extracts were prepared from Sclerotinia-infected and non-infected sunflower plant tissues and from chemically treated sunflower leaf tissues as previously described in Example 1. Northern blot assays were performed for these total RNA samples as described in Example 1 using ³²P-labeled chitinase or LTP cDNA fragments as probes. Results are shown in FIG. 2 (chitinase) and FIG. 3 (LTP).

Northern blot analysis showed that chitinase transcripts were highly induced by Sclerotinia infection. Interestingly, induction occurred in sunflower stem and root but was not observed in petiole or receptacle (FIG. 2). Repression was found in corolla tube and seed tissues. Northern blot analysis also showed that LTP transcripts were induced by Sclerotinia infection in petioles at a late stage. However, LTP transcripts appeared to be present constitutively in stem, corolla tube, and root tissue (FIG. 3).

### EXAMPLE 3

### Expression of Chitinase and LTP in Oxox Transgenic and Non-Transgenic Sunflower

Four-, six-, and eight-week-old non-transgenic SMF3 sunflower plants and oxalate oxidase-transgenic sunflower plants (herein, “oxox”; line 610255) expressing a wheat oxalate oxidase gene were harvested and total RNA extracts prepared as described in Example 1.

Northern blot analysis of these total RNA extracts revealed that steady-state levels of both chitinase mRNA (FIG. 2) and LTP mRNA (FIG. 3) were highly induced in oxox-transgenic leaf tissues starting from about the six-week stage. Steady-state levels of chitinase and LTP transcripts were moderately and highly induced, respectively, in stem tissues of six-week-old oxox transgenic plants.

### EXAMPLE 4

### Transformation and Regeneration of Transgenic Plants

Immature maize embryos from greenhouse donor plants are bombarded with a plasmid containing the chitinase or LTP gene operably linked to a Rsyn7 promoter and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the chitinase or LTP gene operably linked to a Rsyn7 promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

110 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for resistance to Sclerotinia infection.

Bombardment and Culture Media

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite® (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite® (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/i MS vitamins stock solution (0.10 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite® (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### EXAMPLE 5

### Sunflower Meristem Tissue Transformation

Sunflower meristem tissues are transformed with an expression cassette containing the chitinase or LTP gene operably linked to a ubiquitin promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox® bleach solution with the addition of two drops of Tween® 20 per 50 ml of solution. The seeds are rinsed twice with sterile distilled water.

Split embryonic axis explants are prepared by a modification of procedures described by Schrammeijer et al. (Schrammeijer et al (1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar®.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHAL 05 is used in all transformation experiments. A binary plasmid vector comprising the expression cassette that contains the chitinase or LTP gene operably linked to the ubiquitin promoter is introduced into Agrobacterium strain EHA 05 via freeze-thawing as described by Holsters et al. (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/I Bactopeptone, and 5 gm/l NaCl, pH 7.0) with the appropriate antibiotics required for bacterial strain and binary plasmid maintenance. The suspension is used when it reaches an OD600 of about 0.4 to 0.8. The Agrobacterium cells are pelleted and resuspended at a final OD600 of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for chitinase or LTP activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA and/or by chitinase or LTP activity analysis of leaf extracts while transgenic seeds harvested from NPTII-positive To plants are identified by chitinase or LTP activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox® bleach solution with the addition of two to three drops of Tween® 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar® at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar®), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for chitinase or LTP activity using assays known in the art. After positive (i.e., for chitinase or LTP expression) explants are identified, those shoots that fail to exhibit chitinase or LTP activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for chitinase or LTP expression are grafted to Pioneer hybrid 6440 in vitro-grown sunflower seedling rootstock. The rootstocks are prepared in the following manner. Seeds are dehulled and surface-sterilized for 20 minutes in a 20% Clorox® bleach solution with the addition of two to three drops of Tween® 20 per 100 ml of solution, and are rinsed three times with distilled water. The sterilized seeds are germinated on the filter moistened with water for three days, then they are transferred into 48 medium (half-strength MS salt, 0.5% sucrose, 0.3% Gelrite® pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated nucleic acid molecule having a nucleotide sequence for a promoter that is capable of initiating transcription in a plant cell, wherein said nucleotide sequence for said promoter is selected from the group consisting of:
a) a nucleotide sequence comprising the sequence set forth in SEQ ID NO:5 or SEQ ID NO:6;
b) a nucleotide sequence selected from the group consisting of the sequences deposited as Patent Deposit No. PTA-2182;
c) a nucleotide sequence comprising at least 30 contiguous nucleotides of the sequence set forth in SEQ ID NO:5 or SEQ ID NO:6;
d) a nucleotide sequence having at least 70% sequence identity to the nucleotide sequence set forth in SEQ ID NO:5 or SEQ ID NO:6;
e) a nucleotide sequence having at least 80% sequence identity to the nucleotide sequence set forth in SEQ ID NO:5 or SEQ ID NO:6;
f) a nucleotide sequence having at least 90% sequence identity to the nucleotide sequence set forth in SEQ ID NO:5 or SEQ ID NO:6; and
g) a nucleotide sequence that hybridizes under stringent conditions to the complement of a sequence of a), b), or c).

2. A DNA construct comprising a nucleotide sequence of claim 1 operably linked to a heterologous nucleotide sequence of interest.

3. A vector comprising the DNA construct of claim 2.

4. A host cell having stably incorporated in its genome the DNA construct of claim 2.

5. A method for inducing expression of a heterologous nucleotide sequence in a plant, said method comprising the steps of transforming a plant cell with a DNA construct comprising said heterologous nucleotide sequence operably linked to a promoter that is capable of initiating transcription in a plant cell in response to a stimulus, regenerating a stably transformed plant from said plant cell, and exposing said plant to said stimulus, wherein said promoter comprises a nucleotide sequence of claim 1.

6. The method of claim 5, wherein said plant is a monocot.

7. The method of claim 5, wherein said plant is a dicot.

8. The method of claim 7, wherein said dicot is sunflower.

9. A plant cell stably transformed with a DNA construct comprising a heterologous nucleotide sequence operably linked to a promoter that is capable of initiating transcription in said plant cell, wherein said promoter comprises a nucleotide sequence of claim 1.

10. A plant stably transformed with a DNA construct comprising a heterologous nucleotide sequence operably linked to a promoter that is capable of initiating transcription in a plant cell, wherein said promoter comprises a nucleotide sequence selected from the group consisting of:
a) a nucleotide sequence comprising the sequence set forth in SEQ ID NO:5 or SEQ ID NO:6;
b) a nucleotide sequence selected from the group consisting of the sequences deposited as Patent Deposit No. PTA-2182;
c) a nucleotide sequence comprising at least 30 contiguous nucleotides of the sequence set forth in SEQ ID NO:5 or SEQ ID NO:6;
d) a nucleotide sequence having at least 70% sequence identity to the nucleotide sequence set forth in SEQ ID NO:5 or SEQ ID NO:6;
e) a nucleotide sequence having at least 80% sequence identity to the nucleotide sequence set forth in SEQ ID NO:5 or SEQ ID NO:6;
f) a nucleotide sequence having at least 90% sequence identity to the nucleotide sequence set forth in SEQ ID NO:5 or SEQ ID NO:6; and
g) a nucleotide sequence that hybridizes under stringent conditions to the complement of a sequence of a), b), or c).

11. The plant of claim 10, wherein said plant is a monocot.

12. The plant of claim 11, wherein said plant is a dicot.

13. The plant of claim 12, wherein dicot is sunflower.

14. Transformed seed of the plant of claim 10.

15. An isolated nucleic acid molecule having a nucleotide sequence selected from the group consisting of:
a) the sequence set forth in SEQ ID NO:1 or SEQ ID NO:3;
b) a nucleotide sequence selected from the group consisting of the sequences deposited as Patent Deposit No. PTA-2182;
c) a nucleotide sequence encoding the amino acid sequence set forth in SEQ ID NO:2 or SEQ ID NO:4;
d) a nucleotide sequence encoding the amino acid sequence encoded by a nucleotide sequence deposited as Patent Deposit No. PTA-2182;
e) a nucleotide sequence comprising at least 16 contiguous nucleotides of a nucleotide sequence of a), b), c), or d);
f) a nucleotide sequence having at least 70% identity with SEQ ID NO:1, wherein said nucleotide sequence encodes a polypeptide having chitinase activity;
g) a nucleotide sequence having at least 80% identity with SEQ ID NO:1, wherein said nucleotide sequence encodes a polypeptide having chitinase activity;
h) a nucleotide sequence having at least 90% identity with SEQ ID NO:1, wherein said nucleotide sequence encodes a polypeptide having chitinase activity;
i) a nucleotide sequence having at least 70% identity with SEQ ID NO:3, wherein said nucleotide sequence encodes a polypeptide having lipid transfer activity;
j) a nucleotide sequence having at least 80% identity with SEQ ID NO:3, wherein said nucleotide sequence encodes a polypeptide having lipid transfer activity;
k) a nucleotide sequence having at least 90% identity with SEQ ID NO:3, wherein said nucleotide sequence encodes a polypeptide having lipid transfer activity;
l) a nucleotide sequence that hybridizes under stringent conditions to the complement of a sequence of a), b), c), d), or e); and
m) the complement of a nucleotide sequence of a), b), c), d), e), f), g), h), i), j), k), or 1).

16. A DNA construct comprising a nucleotide sequence of claim 15 operably linked to a promoter that drives expression in a plant cell.

17. A vector comprising the DNA construct of claim 16.

18. A host cell having stably incorporated in its genome the DNA construct of claim 16.

19. A method for creating or enhancing disease resistance in a plant, said method comprising transforming said plant with a DNA construct comprising a nucleotide sequence operably linked to a promoter that drives expression of a coding sequence in a plant cell and regenerating stably transformed plants, wherein said nucleotide sequence is selected from the nucleotide sequences of claim 15.

20. The method of claim 19, wherein said plant is a dicot.

21. The method of claim 20, wherein said dicot is sunflower.

22. The method of claim 19, wherein said promoter is an inducible promoter.

23. The method of claim 22 wherein said inducible promoter is selected from the group consisting of promoters for sunflower chitinase and sunflower LTP.

24. A plant cell stably transformed with a DNA construct comprising a nucleotide sequence operably linked to a promoter that drives expression of a coding sequence in a plant cell, wherein said nucleotide sequence is selected from the nucleotide sequences of claim 15.

25. A plant stably transformed with a DNA construct comprising a nucleotide sequence operably linked to a promoter that drives expression of a coding sequence in a plant cell, wherein said nucleotide sequence is selected from the group consisting of:
a) the sequence set forth in SEQ ID NO:1 or SEQ ID NO:3;
b) a nucleotide sequence selected from the group consisting of the sequences deposited as Patent Deposit No. PTA-2182;
c) a nucleotide sequence encoding the amino acid sequence set forth in SEQ ID NO:2 or SEQ ID NO:4;
d) a nucleotide sequence encoding the amino acid sequence encoded by a nucleotide sequence deposited as Patent Deposit No. PTA-2182;
e) a nucleotide sequence comprising at least 16 contiguous nucleotides of a nucleotide sequence of a), b), c), or d);
f) a nucleotide sequence having at least 70% identity with SEQ ID NO:1, wherein said nucleotide sequence encodes a polypeptide having chitinase activity;
g) a nucleotide sequence having at least 80% identity with SEQ ID NO:1, wherein said nucleotide sequence encodes a polypeptide having chitinase activity;
h) a nucleotide sequence having at least 90% identity with SEQ ID NO:1, wherein said nucleotide sequence encodes a polypeptide having chitinase activity;
i) a nucleotide sequence having at least 70% identity with SEQ ID NO:3, wherein said nucleotide sequence encodes a polypeptide having lipid transfer activity;
j) a nucleotide sequence having at least 80% identity with SEQ ID NO:3, wherein said nucleotide sequence encodes a polypeptide having lipid transfer activity;
k) a nucleotide sequence having at least 90% identity with SEQ ID NO:3, wherein said nucleotide sequence encodes a polypeptide having lipid transfer activity;
l) a nucleotide sequence that hybridizes under stringent conditions to the complement of a sequence of a), b), c), d), or e); and
m) the complement of a nucleotide sequence of a), b), c), d), e), f), g), h), i), j), k), or 1).

26. Transformed seed of the plant of claim 25.

27. A substantially purified protein having an amino acid sequence selected from the group consisting of:
a) the amino acid sequence set forth in SEQ ID NO:2 or SEQ ID NO:4;
b) an amino acid sequence encoded by the nucleotide sequence deposited as Patent Deposit No. PTA-2182;
c) an amino acid sequence that shares at least 70% sequence identity to the amino acid sequence set forth in SEQ ID NO:2, wherein said amino acid sequence has chitinase activity;
d) an amino acid sequence that shares at least 70% sequence identity to the amino acid sequence set forth in SEQ ID NO:4, wherein said amino acid sequence has lipid transfer activity;
e) an amino acid sequence encoded by the nucleotide sequence set forth in SEQ ID NO:1 or SEQ ID NO:3; and
f) an amino acid sequence encoded by a nucleotide sequence that hybridizes under stringent conditions to the nucleotide sequence set forth in SEQ ID NO:1, wherein said amino acid sequence has chitinase activity; and
g) an amino acid sequence encoded by a nucleotide sequence that hybridizes under stringent conditions to the nucleotide sequence set forth in SEQ ID NO:3, wherein said amino acid sequence has lipid transfer activity.

28. A composition comprising the protein of claim 27 and a carrier.

29. The composition of claim 28, wherein said carrier is selected from a surface active agent, an inert carrier, an encapsulating agent, and an agrochemical.

30. The composition of claim 28, wherein said carrier is a pharmaceutical carrier.

31. A method for controlling a plant pathogen, said method comprising applying an anti-pathogenic amount of the protein of claim 27 to the environment of said pathogen.

32. The method of claim 31 wherein said anti-pathogenic amount of said protein is applied to a plant.

33. The method of claim 31 wherein said anti-pathogenic amount of said protein is applied by a procedure selected from the group consisting of spraying, dusting, scattering, and seed coating.

34. A method for controlling a plant pathogen comprising applying an anti-pathogenic amount of the composition of claim 28 to the environment of said pathogen.
